(19) United States
(12) Patent Application Publication
(10) Pub. No.: US 2014/0371099 A1
(43) Pub. Date: Dec. 18, 2014

(54) BIOMARKER FOR CHRONIC INFLAMMATORY LUNG DISEASES

(71) Applicants: UNIVERSITE DE STRASBORG, STRASBOURG (FR); CENTRE NATIONAL DE LA RECHERCHE SCIENTIFIQUE, PARIS (FR)

(72) Inventors: Nelly Frossard, Strasbourg (FR); Jacques Haiech, Strasbourg (FR); Christine Lehalle, Illkirch (FR); Romain Kessler, Oberhausbergen (FR); Antoine Magnan, Nantes (FR); Pascal Chanez, Marseille (FR); Jean-Luc Galzi, Weitbruch (FR)

(21) Appl. No.: 14/375,502

(22) PCT Filed: Jan. 31, 2013
(86) PCT No.: PCT/EP2013/051964

§ 371 Date: Jul. 30, 2014

(30) Foreign Application Priority Data
Jan. 31, 2012 (FR) 1250917

(51) Int. Cl. C12Q 1/68; G01N 33/68

(52) U.S. Cl. CPC C12Q 1/6881; G01N 33/6884; C12Q 2600/158; G01N 2800/122; G01N 2333/7158
USPC 506/9; 435/6.12; 435/7.24

(57) ABSTRACT

The invention relates to an in vitro method for the diagnosis and/or prognosis and/or evaluation of the progression of a chronic inflammatory lung disease in a subject, using the level of expression of the GPR15 G protein-coupled receptor gene as a biomarker for the disease.

The invention relates to the use of a novel biomarker for diagnosing a chronic inflammatory lung disease, specifically asthma and chronic obstructive pulmonary disease (COPD) in an individual. The biomarker according to the invention also makes it possible to determine the susceptibility of a subject to developing a chronic inflammatory lung disease, and/or to monitor the progression of a chronic inflammatory lung disease in an affected subject. More particularly, the biomarker makes it possible to differentiate asthma and COPD.

### CONTEXT OF THE INVENTION

Chronic inflammatory lung diseases, which affect the respiratory system to varying degrees, have been greatly increasing for several decades, in particular in industrialized countries. Nowadays, the number of deaths throughout the world related to these diseases is estimated at more than 3 million per year. The main environmental factors in the dock are smoking and poor air quality. Thus, in industrialized countries, the increase in the air of chemical pollutants contributes to the emergence of new cases and to the worsening of the already listed cases. Among these chronic inflammatory diseases of the respiratory tracts are principally asthma and chronic obstructive pulmonary disease, which however exhibit opposite inflammatory profiles.

Asthma is the chronic inflammatory pulmonary disease which is the most widespread in industrialized countries and which affects all age categories. Asthma is a multifactorial disease which does not necessarily have a genetic origin. It is characterized mainly by inflammatory bronchia, contributing to a reduction in their diameter and thus making it difficult for asthmatic individuals to breathe out during breathing. This inflammation is reversible, such that the asthmatic individual can, outside the asthma attacks, show no particular symptom of the disease. Depending on the cause, the number and frequency of the asthma attacks, the terms intermittent asthma, mild persistent asthma, moderate persistent asthma and severe persistent asthma are used. In addition, depending on the nature of the asthma, chronic, allergic, exercise-induced, etc., the diagnosis can be more difficult to establish. Generally, it is only after the occurrence of repeated asthma attacks that a thorough examination of the respiratory/ventilatory function of the patient's lungs is carried out. The associated tests, such as the bronchial hyperreactivity test or the bronchiodilatator reversibility test, are trying for the patient and the results can sometimes be difficult for the care staff to interpret, depending on the nature of the asthma, on the state of the disease, on the patient's state of physical health at the time of the examination, etc.

Chronic obstructive pulmonary disease (COPD), for its part, groups together several chronic diseases of the respiratory system, including chronic bronchitis and emphysema. COPD is characterized by a decrease in the expiratory flow due mainly to a bronchial obstruction that is not completely reversible with a beta2 adrenergic agonist and which gradually worsens. COPD is one of the most widespread pathological conditions associated with smoking and affects between 4% and 10% of the worldwide adult population, with a greater prevalence in adults over the age of 40. Generally, the symptoms of COPD include coughing, expectoration and increasing breathlessness during exercise. The diagnosis of severity is based on a function respiratory test which measures the degree of bronchial obstruction. Currently, screening for COPD via functional examination is limited, since this examination is not very accessible, and the proportion of undiagnosed afflicted patients is estimated at more than 40%.

Moreover, the differential diagnosis of asthma and COPD often proves to be quite complex. The result of the bronchial reversibility test with a mimetic beta-2 agonist is not always clear. However, it is important to clearly distinguish between these two pathological conditions for the choice of the appropriate treatment, and also for the characterization of patients entering into clinical trials.

Faced with the constraint and the difficulty of these chronic inflammatory lung disease diagnoses, the development of effective reliable biomarkers would make it possible to diagnose these diseases more easily, to screen for them at an early stage, to establish a prognosis thereof and to improve the treatment of patients by giving them the appropriate treatment as early as possible. The benefit of such biomarkers would be i) rapid indication of the appropriate treatment in normal clinical practice (societal advantage), and ii) a finer characterization of patients entering into clinical trials for new molecules or for new therapeutics (industrial advantage).

### SUMMARY OF THE INVENTION

The present invention aims to overcome the difficulties associated with the screening and diagnosis of chronic inflammatory lung diseases and with the monitoring of the progression of the disease in patients, by proposing the use of a specific biomarker, the expression profile of which is representative of these diseases. In particular, the present invention is of use for the differential diagnosis of asthma and COPD. Alternatively, the present invention is of use for the diagnosis of asthma. Moreover, it is also of use for the diagnosis of COPD.

The inventors have demonstrated, unexpectedly, that the analysis of the expression of the gene of a G protein-coupled receptor, GPR15, is particularly suitable for the characterization of chronic inflammatory lung diseases, and quite particularly asthma and COPD. The analysis of the GPR15 gene expression profile makes it possible to rapidly and reliably establish, independently of factors external to the disease, whether a subject is suffering from one or another chronic inflammatory lung disease: the inventors have discovered that patients exhibit a different GPR15 gene expression profile depending on whether they are suffering from asthma or from COPD. The biomarker according to the invention therefore makes it possible to determine whether a patient is suffering from asthma or from COPD. The use of the GPR15 gene expression profile according to the invention makes it possible to establish a diagnosis from a simple biological sample, and in particular a blood sample from the patient, thereby making such a diagnosis simple and inexpensive. In addition, the monitoring over time of the expression profile of the biomarker according to the invention in a patient suffering from a chronic inflammatory lung disease makes it possible to study the progression of the disease with or without treatment, or to forecast its progression. In particular, the biomarker could be used to determine the stage of the disease. Finally, the use of the GPR15 gene expression profile according to the invention makes it possible to select the most suitable treatment for the patients and/or to select the patients requiring a particular clinical follow-up.

Thus, a subject of the invention is an in vitro or ex vivo method for the diagnosis and/or prognosis and/or evaluation of the progression of a chronic inflammatory lung disease in a subject, according to which the GPR15 gene expression level is measured in a biological sample from the subject.

In one particular example of the invention, the method is implemented for diagnosing/forecasting/evaluating the progression of asthma in a subject, an underexpression of the GPR15 gene being indicative of asthma.

In another particular example of the invention, the method is implemented for diagnosing/forecasting/evaluating the progression of COPD in a subject, an overexpression of the GPR15 gene being indicative of COPD. Thus, the present invention relates to an in vitro method for the diagnosis of COPD in a subject, in which the G protein-coupled receptor 15 (GPR15) gene expression level is measured in a biological sample from the subject and the GPR15 gene expression level is compared to a reference expression level taking into account the age of the patient, an overexpression of GPR15 being indicative of COPD.

Thus, the present invention relates to an in vitro method of diagnosis which makes it possible to determine whether a patient is suffering from asthma or from chronic obstructive pulmonary disease (COPD), in which the G protein-coupled receptor 15 (GPR15) gene expression level is measured in a biological sample from the patient, an underexpression of GPR15 being indicative of asthma and an overexpression of GPR15 being indicative of COPD.

The GPR15 gene expression level in the biological sample from the subject for whom a diagnosis/prognosis is to be provided is compared to a reference expression level. Preferably, the reference expression level takes into account the age of the patient.

The present invention relates to an in vitro method for the diagnosis of asthma and/or COPD in a subject, in which the GPR15 gene expression level is measured in a biological sample from the subject and the GPR15 gene expression level is compared to a reference expression level taking into account the age of the patient, an overexpression of GPR15 being indicative of COPD and an underexpression of GPR15 being indicative of asthma.

According to the invention, the GPR15 gene expression level is advantageously measured at the nucleic and/or protein level, for example by measuring the amount of mRNA transcribed and/or by measuring the amount of GPR15 protein using at least one specific method for measuring GPR15 expression. The techniques which make it possible to detect the expression of the GPR15 gene at the nucleic level are well known to those skilled in the art. The detection can in particular be carried out by real-time quantitative RT-PCR, a microfluidic technique, a DNA chip, high-throughput mRNA sequencing, or any appropriate mRNA quantification technique, such as an RNA chip or the LCR (“Ligase Chain Reaction”), TMA (“Transcription Mediated Amplification”), PCE (“enzyme amplified immunoassay”) and bDNA (“branched DNA signal amplification”) methods, etc. The techniques which make it possible to detect the expression of the GPR15 gene at the protein level are also well known to those skilled in the art and can in particular include flow cytometry, quantitative immunocytochemistry, cellular ELISA, the Taqman® protein assay (Taqman® Protein Assay, Applied Biosystems), protein or antibody chips optionally coupled to mass spectrometry, the binding of labelled ligands, etc.

According to one example of implementation of the invention, the biological sample is a whole blood sample, the detection of the GPR15 gene expression product preferably being carried out on the total leukocyte population.

Another subject of the invention relates to the use of a kit comprising at least one reagent specific for the GPR15 gene expression product, preferably chosen from a monoclonal or polyclonal antibody or any other type of molecules or macromolecules capable of interacting specifically with the GPR15 protein, a natural or synthetic ligand, or a nucleic sequence capable of hybridizing specifically with a fragment of the mRNA encoding GPR15, for the diagnosis and/or the prognosis and/or the evaluation of the progression of a chronic inflammatory lung disease in a subject. The chronic inflammatory lung disease may be asthma or COPD. The invention relates to the use of a kit comprising at least one reagent specific for the GPR15 gene expression product for the diagnosis of asthma or the differential diagnosis of asthma and COPD in a subject. The present invention also relates to the use of a kit comprising at least one reagent specific for the GPR15 gene expression product and informations relating to reference GPR15 expression levels taking into account age, for the diagnosis of COPD in a subject.

A subject of the invention is also the in vitro or ex vivo use of a GPR15 gene expression product as a biomarker for the diagnosis and/or the prognosis and/or the evaluation of the progression of a chronic inflammatory lung disease in a subject. Preferably, the chronic inflammatory lung disease is asthma or COPD. In addition, the invention relates to the in vitro or ex vivo use of a GPR15 gene expression product as a biomarker for the differential diagnosis of asthma and COPD in a subject.

### BRIEF DESCRIPTION OF THE FIGURES

FIG. 1: Diagrammatic representation of the decision tree having resulted in the selection of GPR15 as a biomarker for chronic inflammatory lung diseases.

FIG. 2: Boxplot showing the GPR15 gene expression profile in patients suffering from asthma or from COPD compared with the expression profile in control volunteers who do not present these diseases.

FIG. 3: GPR15 gene expression in patients suffering from asthma or from COPD versus controls who do not present these diseases, represented as amplification threshold cycles (Ct) as a function of age.

### DETAILED DESCRIPTION OF THE INVENTION

The present invention is based on the study of the expression profile of the G protein-coupled receptor 15 [GPR15, GPCR15, or BOB (Brother of Bonzo)] gene.

The G protein-coupled receptor family groups together many membrane receptors, including GPR15. The gene encoding GPR15 is located on chromosome 3q11.2-q13.1 in humans (Heiber et al, Genomics 32, 462-465, 1995). To date, a link between GPR15 and infection by the human immunodeficiency virus (HIV) has been demonstrated (Blaak et al, Journal of virology, 79: 1686-1700, 2005,—Okamoto & Shikano, Journal of Biological Chemistry 286, 7171-7181, 2001). GPR15 is thought to be a coreceptor for HIV entry into circulating lymphocytes.

As used in the present document, the term “GPR15” refers to the G protein-coupled receptor 15. It is described in the gene databases under the following references: HGNC:4469; GeneID:2838; RefSeq Protein:NP 005281.1; RefSeq ARN:NM_{—}005290.1. Of course, GPR15 relates to that of human beings.

Surprisingly, the inventors have demonstrated a correlation between GPR15 expression and chronic inflammatory lung diseases. More particularly, the inventors have discovered that the analysis of the GPR15 gene expression profile makes it possible to reliably determine whether a subject is suffering from a chronic inflammatory lung disease, and advantageously to differentiate subjects suffering from asthma from subjects suffering from COPD.

Thus, the invention proposes to use a GPR15 gene expression product as a biomarker, in particular for diagnosing a chronic inflammatory lung disease, but also for the prognosis and the monitoring of the progression of a chronic inflammatory lung disease. Preferably, the chronic inflammatory lung disease is asthma or COPD. In particular, it proposes to use a GPR15 gene expression product as a biomarker for the differential diagnosis of asthma and COPD, i.e. for differentiating subjects suffering from asthma from subjects suffering from COPD.

More particularly, the invention proposes an in vitro or ex vivo method for the diagnosis and/or prognosis and/or evaluation of the progression of a chronic inflammatory lung disease in a subject, according to which the G protein-coupled receptor 15 (GPR15) gene expression level is measured in a biological sample from the subject.

In the context of the present invention, a “subject” or a “patient” includes any mammalian subject or patient, and preferably a human subject or patient, including in particular children and adults.

The term “chronic inflammatory lung diseases” is intended to mean chronic inflammatory diseases of the respiratory tracts, and more specifically asthma and chronic obstructive pulmonary disease, including chronic bronchitis and emphysema.

In the context of the invention, the object of the “diagnosis” is to detect and/or identify, in a subject, a chronic inflammatory lung disease, whatever its stage. In particular, the diagnosis makes it possible to determine whether a subject is suffering from asthma or from COPD.

The “prognosis” is understood to be the evaluation of the disease with regard to the susceptibility of developing the disease, and/or with regard to the susceptibility of progression towards a more advanced stage/degree, and/or with regard to the risks of complications and of exacerbations, and/or with regard to its outcome, etc.

The “evaluation of the progression of the disease” corresponds to the analysis over time of the progression of the disease which was previously diagnosed or for which a prognosis was previously provided. Such monitoring over time may make it possible to select, validate and/or adapt a treatment. It also makes it possible to determine the intensity of the clinical follow-up required for the patient. This follow-up makes it possible to determine at any moment whether a treatment is necessary.

The invention also proposes a method for providing information which may be of use for diagnosing and/or forecasting and/or evaluating the progression of a chronic inflammatory lung disease in a subject, according to which the GPR15 gene expression level is measured in a biological sample from the subject. Preferably, the chronic inflammatory lung disease is asthma or COPD. More particularly, the invention proposes a method for providing information which may be of use for differentially diagnosing asthma or COPD in a patient, i.e. for differentiating subjects suffering from asthma from subjects suffering from COPD. According to the invention, GPR15 can be used as a biomarker for the prognosis and/or the diagnosis of a chronic inflammatory lung disease, and more specifically COPD and asthma, and also for the differential diagnosis of asthma and COPD.

Likewise, GPR15 can be used as a biomarker for monitoring a subject diagnosed as having a chronic inflammatory lung disease and/or forecast as liable to develop such a disease and/or to develop such a disease with a more advanced/severe stage and/or to develop complications and/or exacerbations. Preferably, the chronic inflammatory lung disease is asthma. Alternatively, the chronic inflammatory lung disease is COPD.

Such a biomarker can also be used for the selection of subjects suffering from a chronic inflammatory lung disease capable of benefitting from a particular treatment and/or for evaluating the progression of the disease in subjects suffering from a chronic inflammatory lung disease during a treatment. GPR15 can thus serve as a biomarker for the selection, evaluation and adaptation of the treatment of a subject suffering from a chronic inflammatory lung disease. The invention is quite particularly of use for choosing the appropriate treatment according to whether the patient is suffering from asthma or from COPD.

According to the invention, GPR15 is used as a biomarker for chronic inflammatory lung diseases, and more specifically asthma and COPD.

The GPR15 gene expression profile can be produced in various ways, in particular by measuring, by determining or by assaying the amount of the expression product of this gene from a biological sample from the subject. The sample is preferably a cell sample originating from a biological fluid from the subject, such as blood. In one preferred embodiment, the sample from the subject is a sample containing leukocytes. The methods according to the present invention can comprise a sampling step and/or a step of preparing/purifying the sample.

The GPR15 gene expression product corresponds to a transcribed or translated product of this gene, such as its mRNA or the G protein-coupled receptor GPR15 itself

The amount of the GPR15 protein can be determined by any method known to those skilled in the art. Conventionally, these methods comprise a step of bringing the sample into contact with a selective ligand of the GPR15 protein, such as an antibody directed against GPR15-specific epitopes, or a fragment or a derivative of this antibody, or an endogenous ligand or a small molecule which binds to the receptor with strong affinity.

Generally, the anti-GPR15 antibodies used in the present invention are antibodies or any protein molecule or the like which binds specifically to GPR15 with a submicromolar affinity. They are, for example, monoclonal antibodies or monospecific polyclonal antibodies, i.e. antibodies which specifically recognize only one epitope. Preferably, the antibodies will be specific for the extracellular part of the receptor.

These antibodies can in particular be obtained by any conventional method of production. In particular, an anti-GPR15 antibody can be obtained by immunization of a non-human animal (rabbit, mouse, etc.) with GPR15 or with an antigenic sequence thereof, sampling and then depletion of the antiserum obtained, for example on an immunoadsorbent containing GPR15, according to methods known to those skilled in the art.

The amount of GPR15 protein can be measured using flow cytometry techniques, immunological assays (ELISA, EIA, RIA, etc.), radioimmunological, chemiluminescent or fluorescent assays, immunoelectrophoreses, immunoprecipitations, the use of mass spectrometry, etc. The method most commonly comprises a step of revealing the labeling, such as fluorescent, radioactive or enzymatic labeling, or using a coloured molecule, or more generally any labeling which makes it possible to demonstrate the formation of the antigen/antibody complex or any biophysical method which makes it possible to demonstrate the interaction between GPR15 and either a protein molecule or a synthetic molecule (aptamer, ligand, etc.).

According to the invention, the mRNAs of the GPR15 gene can be detected by any method known to those skilled in the art. Generally, these methods require a first step of extracting the nucleic acids contained in the biological sample, by means of techniques well known to those skilled in the art, using for example lysing enzymes, appropriate chemical solutions or specific extraction resins. Said mRNAs are then brought into contact with an advantageously labelled nucleic acid probe, specific for GPR15 mRNAs, under conditions which allow hybridization with said mRNAs, followed by assaying of the advantageously labelled hybrid forms obtained. The mRNAs extracted can also be preamplified (for example using the RT-PCR technique). RT-PCR (“reverse transcription polymerase chain reaction”) techniques, and in particular real-time quantitative RT-PCR, are particularly preferred. Alternatively, other methods for detecting nucleic acids can be used, such as the LCR (“Ligase Chain Reaction”), TMA (“Transcription Mediated Amplification”), PCE (“enzyme amplified immunoassay”), bDNA (“branched DNA signal amplification”) and high-throughput sequencing methods. Typically, the detection of the RNA of the GPR15 gene is carried out by:
Obtaining of total or messenger-RNAs from a biological sample, such as a whole blood sample,
cDNA synthesis by reverse transcription,
PCR amplification of the cDNA in the presence of oligonucleotide primers specific for the GPR15 gene,
quantification of the PCR product, in particular by bringing into contact with an advantageously labelled nucleic acid probe specific for the GPR15 mRNAs (indirect detection), in particular under conditions which allow hybridization, followed by assaying of the advantageously labelled, hybrid forms obtained.

According to one particular embodiment of the invention, the expression profile of a GPR15 gene product in a biological sample from a patient is compared to the reference expression profile of a GPR15 gene product. This reference expression profile may be the one obtained from a control biological sample. Of course, the control expression profile may correspond to a mean or a median of the expression profiles obtained for a panel of control biological samples. The expression levels obtained in the samples from the subjects to be analysed and in the control samples are advantageously standardized using the expression level of proteins known to have a stable expression level in the subjects with a chronic inflammatory lung disease and in the healthy subjects, such as HPRT1 (hypoxanthine-guanine phosphoribosyltransferase 1), TBP (TATA binding protein) or TFRC (Transferrin receptor protein 1) for human leukocyte mRNA, and such as β-actin or GAPDH (glyceraldehyde-3-phosphate dehydrogenase) for the protein samples.

Preferentially, the GPR15 gene products analysed for producing the patient's expression profile in a biological sample and for producing the control expression profile are identical, as is the nature of the samples used. They can, however, comprise possible mutations that will be demonstrated by sequencing.

In one example of implementation, the reference expression profile is produced from biological samples from subjects who are not liable to have a chronic inflammatory lung disease. Such a reference expression profile makes it possible in particular to diagnose a chronic inflammatory lung disease at a very early stage of the disease.

In another example of implementation, the reference expression profile is produced from biological samples from subjects who have a diagnosed chronic inflammatory lung disease, and the stage/degree of which is advantageously known. It may be particularly advantageous to produce reference expression profiles from biological samples from subjects diagnosed as having a chronic inflammatory lung disease, for each of the stages of the disease. Such profiles may in particular be of use for monitoring the progression of the disease in a subject, in order in particular to evaluate the risks of complication and/or of exacerbation, and/or to select, adapt, modify, begin or stop a treatment.

For example, in the case of asthma, it is possible to produce reference expression profiles from subjects diagnosed as having asthma described as “controlled” or “non-controlled” according to the GINA (“global initiative for asthma”) classification (2006, 2011), or according to the stages of severity: intermittent or mild, moderate or severe persistent (GINA 2002). The asthma may also be described according to promoting factors such as allergy, the terms “allergic asthma” or “non-allergic asthma” are then used.

Likewise, in the case of COPD, it is possible to produce reference expression profiles from subjects diagnosed as having chronic bronchitis, from subjects diagnosed as having emphysema, and from subjects diagnosed as having chronic bronchitis and emphysema. It is also possible to take into account the degree of bronchial obstruction (Tiffeneau index: FEV1/FVC<70%). For example, it is possible to produce reference expression profiles from subjects who have mild (FEV1≧80%), moderate (50%≦FEV1≦80%), severe (30%≦FEV1≦50%) and very severe (FEV1<30%) chronic bronchitis.

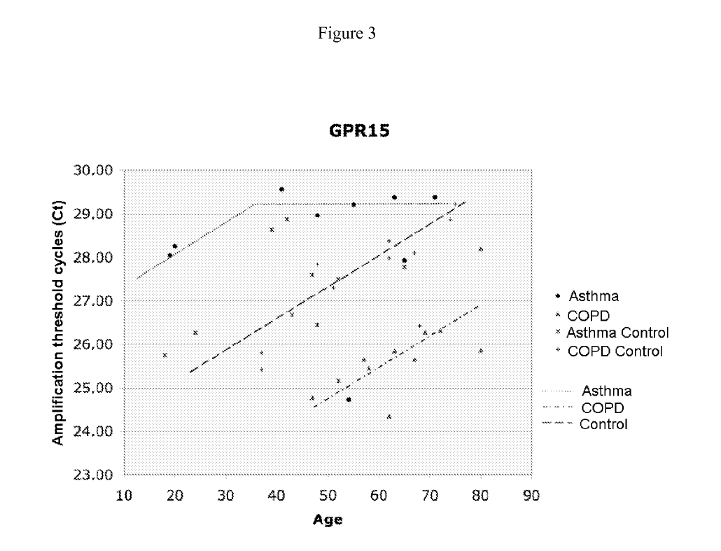

Surprisingly, the inventors have demonstrated a correlation between the GPR15 expression level and the age of the subject. This observation has been extended to all patients suffering from asthma, those suffering from COPD and for all the control subjects. More particularly, as shown in FIG. 3, the patients suffering from asthma exhibit low GPR15 expression levels (in particular, a high number of amplification cycles is required in order to be detected) which are constant according to age. Conversely, it has been observed that the control (healthy) subjects and the COPD subjects exhibit a GPR15 expression level which decreases according to age. For COPD, the inventors have demonstrated that the age parameter must absolutely be taken into account. Thus, preferentially, the reference expression level takes into account the age of the patient. However, it may also be noted that, for differentiating between COPD and asthma, the subject's age parameter can optionally be neglected since the two groups are sufficiently distinct from one another. Reference expression profiles can therefore be produced as a function of the age of the control subjects. The GPR15 expression level for a patient is then advantageously compared to the reference level of controls of the same age category (patient's age±2.5 years).

Since the symptoms of the disease appear late for patients suffering from COPD (>40 years old) and earlier for asthmatic patients, the age of the control population preferentially rises in tiers from 18 to 85 years old.

Such a classification according to age category can be particularly advantageous for the diagnosis or the prognosis of chronic lung diseases known to appear at a particular age. This classification can also prove to be of use for evaluating the progression of the disease, and in particular for COPDs, the progression of which according to the patient's age can be rapid and/or critical.

The various criteria set out above for producing control expression profiles are not exclusive with respect to one another. For example, it may be particularly advantageous to produce reference expression profiles according to the stage of the disease and the age of the subjects.

The inventors have discovered that patients suffering from asthma exhibit a GPR15 gene expression profile opposite to the expression profile of patients suffering from COPD. Thus, the use of the GPR15 biomarker proves to be particularly relevant for reliably determining whether a subject is suffering from one or another of these two chronic lung diseases.

More specifically, the subjects suffering from asthma exhibit a decrease in GPR15 gene product expression compared with the GPR15 gene product expression in a control subject not suffering from this disease. Conversely, the subjects suffering from COPD exhibit an overexpression of the GPR15 gene product compared with the expression of the GPR15 gene product in a control subject not suffering from this disease.

In one example of implementation of the invention, the method comprises a step for determining whether the GPR15 gene product expression level in a subject is high or low compared with the reference expression level.

In one example of implementation, it is considered that the reference expression level corresponds to a normal expression level of GPR15 gene product, i.e. a level outside any chronic inflammatory lung disease. The expression level in the subject is considered to be high, and therefore exhibits an overexpression, if said level is, for example, at least 20%, 30%, 40% or 50% higher than the reference expression level after standardization, and preferentially at least 100%, 150% or 200% higher. Conversely, the expression level in the subject is considered to be low, and therefore exhibits an underexpression, if said level is, for example, at least 20%, 30%, 40% or 50% lower than the reference expression level after standardization, and preferably at least 100%, 150% or 200% lower. The patient's own GPR15 gene product expression level at a given time t can serve as reference expression level for monitoring the progression of the disease of said patient at a time t+1. An overexpression or an underexpression of the patient's GPR15 gene product at t+1 compared with the patient's GPR15 gene product expression level at the time t can be indicative of an improvement or of a degradation of the state of health of said patient according to the disease observed.

In another example of implementation, it is considered that the reference expression level corresponds to a GPR15 gene product expression level for a population of patients suffering from asthma for the diagnosis of asthma or from COPD for the diagnosis thereof. Thus, reference curves or clouds will be defined for asthma and/or COPD as a function of age. Once the expression of the GPR15 gene product for the subject to be diagnosed has been determined, said expression will be compared to these reference curves or clouds while taking into account the age of the patient, and it may be determined whether the subject is suffering from COPD or from asthma.

Moreover, it is proposed to use the GPR15 gene expression profile to determine the stage of a chronic lung disease, in particular COPD. Thus, GPR15 gene expression references would be determined for each stage of COPD. Preferably, these GPR15 gene expression references take into account the stage of the COPD and the age of the patient. Thus, the present invention relates to an in vitro method for determining the stage of COPD in a subject, in which the GPR15 gene expression level is measured in a biological sample from the subject, the GPR15 gene expression level is compared to reference expression levels taking into account the stage of the COPD and preferably the age of the subject, and on the basis of this comparison, the stage of the COPD in the subject is determined. A patient's information will be placed in the context of a set of reference data thus making it possible to determine the state of the pathological condition of the patient under consideration. The particular embodiments described above are applicable in this method.

Another subject of the invention is the use of a kit comprising at least one reagent specific for the GPR15 gene expression product, chosen from a monoclonal or polyclonal antibody directed against GPR15 or any protein molecule or the like which binds specifically to GPR15 with a submicromolar affinity, a probe capable of hydridizing specifically with a fragment of the mRNA or of the cDNA encoding GPR15, and at least one pair of nucleic acid primers specific for the mRNA or for the cDNA encoding GPR15, for the diagnosis and/or the prognosis and/or the evaluation of the progression of a chronic inflammatory lung disease in a subject. Preferably, the chronic inflammatory lung disease is asthma or COPD. In addition, the invention relates to the use of a kit comprising at least one reagent specific for the GPR15 gene expression product, for the differential diagnosis of asthma and COPD in a subject.

In one particular embodiment, the invention relates to the use of a kit comprising at least one reagent specific for the GPR15 gene expression product and information relating to reference GPR15 expression levels taking into account age, for the diagnosis of COPD. In particular, the information may be a reference curve/cloud of GPR15 expression in control individuals as a function of age, and/or a reference curve/cloud of GPR15 expression in asthmatic subjects as a function of age, and/or a reference curve/cloud of GPR15 expression in subjects suffering from COPD as a function of age.

In one additional embodiment, the invention relates to the use of a kit for determining the stage of COPD, comprising at least one reagent specific for the GPR15 gene expression product and information relating to reference GPR15 expression levels taking into account the stage of the COPD and optionally age. In particular, the information may be a reference curve/cloud of GPR15 expression in individuals suffering from COPD as a function of the stage of the disease and optionally of age. A patient's information will be placed in the context of a set of reference data thus making it possible to determine the state of the pathological condition of the patient under consideration.

In one particular example of implementation of the invention, the kit may comprise a means for detecting the complex formed between the GPR15 protein and the ligand, and/or a means for detecting the hybridization of at least one specific probe to a fragment of the mRNA or the cDNA encoding GPR15 and/or means for amplifying and/or detecting said mRNA or cDNA.

Other aspects and advantages of the present invention will become apparent in the following experimental section, which should be regarded as an illustration, which does not in any way limit the scope of the protection sought.

EXPERIMENTATIONS

### Material and Method

### Patient Characteristics

The asthmatic patients and their genetically paired controls (ascendant, descendant, siblings) are recruited in the pneumology departments of the Nantes hospital (Prof. A. Magnan) and the Marseilles hospital (Prof P. Chanez). The characteristics of these volunteers are described in Table 1 below.

TABLE 1
Characteristics of asthmatic patients and their related controls

| | | | | Length of | | | | | |
|---|---|---|---|---|---|---|---|---|---|
| | | | Smoker | time | | | | | |
| Subject | | | (packets- | (number of | Stage of | Basic | PD20 | Latest | |
| No. | Sex | Age | years) | years) | severity | FEV1% | (μg MCh) | exacerbation | Phadiatop |
| A101 | F | 65 | NS | 20 | Severe | 75 | ND | 20/04/08 | Negative |
| A102 | F | 48 | EX 1 | 20 | Severe | 45 | ND | 1/05/08 | Negative |
| A1002 | F | 71 | NS | 51 | Severe | 53 | ND | Numerous | Negative |
| A1003 | M | 19 | NS | 18 | Severe | 65 | ND | 5 during the | Pos (der.p) |
| | | | | | | | | previous year | |
| A1004 | F | 41 | NS | 23 | Severe | 55 | ND | 6 during the | Positive |
| | | | | | | | | previous year | |
| A1005 | M | 20 | NS | 19 | Severe | 100 | Positive | None | Positive |
| A1006 | F | 54 | S | 19 | Severe | 72 | ND | 10 during the | Negative |
| | | | | | | | | year | |
| A3006 | F | 63 | NS | 63 | Severe | 67 | ND | 20/06/10 | Negative |
| A3007 | F | 55 | NS | 33 | Severe | 79 | ND | 30/03/09 | Positive |
| | | | | | | | | | (Cat IgE) |
| C101 | M | 39 | NS | | | 100 | ND | | Negative |
| C102 | F | 48 | EX 20 | | | 100 | >500 | | Negative |
| C1001 | M | 52 | NS | | | 103 | >3100 | | Negative |
| C1003 | M | 47 | EX | | | 97 | >3100 | | Negative |
| C1004 | F | 18 | ? | | | 114 | >3100 | | Negative |
| C1005 | F | 52 | EX 20 | | | 100 | Negative | | Negative |
| C1006 | F | 24 | S 10 | | | 102 | >500 | | Negative |
| C3004 | F | 42 | NS | | | 90 | >1600 | | Negative |
| C3005 | M | 43 | S 10 | | | 104 | >1600 | | Negative |
| C3006 | M | 65 | NS | | | 76 | >1600 | | Negative |

F = Female;
M = Male;
S = Smoker;
NS = Non-Smoker;
Ex = Ex-Smoker;
ND = Not Determined
FEV1 = Forced expiratory volume in 1 second.
PD20 = Dose of methacholine provoking a 20% drop in FEV1.
Latest exacerbation: latest hospitalization due to a respiratory infection relating to an amplification of the symptoms of the disease
Phadiatop: allergic evaluation test

The patients suffering from chronic obstructive pulmonary disease or COPD and their paired environmental controls (spouse, neighbour) are recruited in the pneumology department of the university hospitals of Strasbourg (Prof R. Kessler). The characteristics of these volunteers are described in Table 2 below.

TABLE 2
Characteristics of COPD patients and their paired environmental controls

| | | | | Length of | | | | | | |
|---|---|---|---|---|---|---|---|---|---|---|
| | | | Smoker | time | | | | | | |
| Subject | | | (packets- | (number of | FEV1 | FEV1/ | Stage of | PD20 | Latest | |
| No. | Sex | Age | years) | years) | (%) | FVC (%) | severity | (μg MCh) | exacerbation | Phadiatop |
| B2003 | M | 67 | S 40 | 9 | 65 | 46 | Moderate | | Sep-08 | ND |
| B2004 | M | 69 | EX 80 | 1 | 61 | 38 | Moderate | | June-08 | Negative |
| B2005 | M | 80 | Ex 10 | 7 | 42 | 52 | Severe | | 2007 | Negative |
| B2006 | F | 57 | S 40 | 2 | 89 | 62 | Moderate | | Nov-09 | Positive |
| B2007 | F | 47 | EX 30 | 3 | 66 | 51 | Moderate | | July-09 | Negative |
| B2009 | F | 80 | S 90 | ND | 59 | 62 | Moderate | | Oct-09 | Negative |
| B2010 | M | 72 | EX 50 | 1 | 51 | 41 | Moderate | | feb-2010 | Positive |
| B2014 | F | 58 | S 30 | 4 | 70 | 61 | Moderate | | 2009 | Negative |
| B2015 | M | 62 | S 40 | 2 | 57 | 41 | Moderate | | ND | Negative |
| B2016 | F | 63 | EX 80 | 3 | 75 | 54 | Moderate | | Jun. 07, 2007 | Negative |
| C2001 | F | 75 | Passive | | 141 | 84 | | >1500 | | ND |
| C2002 | F | 37 | S 20 | | 121 | 87 | | >3100 | | ND |
| C2003 | F | 67 | Passive | | 99 | 82 | | >2080 | | ND |
| C2004 | F | 62 | Passive | | 115 | 83 | | >3100 | | Negative |
| C2006 | F | 51 | EX 30 | | 99 | 86 | | >3100 | | Negative |
| C2007 | F | 48 | NS | | 105 | 88 | | >3100 | | Negative |
| C2008 | F | 62 | NS | | 121 | 87 | | >3100 | | Negative |
| C2010 | F | 68 | S 30 | | 129 | 78 | | >1500 | | Negative |
| C2014 | M | 37 | S 15 | | 96 | 77 | | >3100 | | Negative |
| C2016 | F | 74 | NS | | 115 | 74 | | >3100 | | Negative |

F = Female;
M = Male;
S = Smoker;
NS = Non-Smoker;
Ex = Ex-Smoker;
Passive = Passive smoking
FEV1 = forced expiratory volume in 1 second.
Tiffeneau index: FEV1/FVC (forced vital capacity) ratio as percentage. In an obstructive
patient: FEV1/FVC <70%
PD20 = Dose of methacholine causing a 20% drop in FEV1.
Stage of severity = moderate stage: 80 to 50% of the theoretical value of the FEV1;
severe stage: 50 to 30% of the theoretical value of the FEV1.
Latest exacerbation: latest hospitalization due to a respiratory infection related to an amplification of the symptoms of the disease
Phadiatop: test for evaluating sensitization to common pneumoallergens

The study was carried out after submission to the French Biomedical Research Ethic Committee (CCPPRB) Alsace No. 1 dated 14 Jun. 2005, in accordance with modified Huriet law No. 88-1138 of 20 Dec. 1988 of the public health code, and was declared to the Haute Autorité de Santé [French National Health Authority]. The amendments to the protocol (promotion, additional investigators) were referred to the Ethics Committee (CPP Est No. IV).

### Obtaining Leukocytes

The total leukocytes are obtained from whole blood (20 ml) taken on EDTA. Six ml of blood are injected through a LeukoLOCK™ microporous filter (Fractionation & Stabilization Kit, Life Technologies™) which retains the leukocytes. The filter is rinsed with PBS, then RNAlater® for preservation of the RNAs, and stored at −80° C. until the RNA extraction.

### RNA Extraction and Purification

After thawing, the RNAlater® solution is eliminated by injecting air into the filter using a syringe. A Lysing/Binding solution concentrate (Total RNA isolation Kit. LeukoLOCK™) for lysis of the cells is added to the filter. The lysate is recovered in a 15 ml Falcon tube and treated with the proteinase K from the kit. The RNAs are attached to beads by adding RNA Binding Beads and centrifuged. The supernatant is removed, and the beads are washed 3 times with a solution containing isopropanol and then with ethanol. The RNAs are then eluted (50 μl; elution solution, LeukoLOCK™) into an Eppendorf tube which can be stored at −80° C.

The total RNAs are purified by centrifugation on silica gel which has a high affinity for RNA (RNeasy® mini kit, Qiagen), eluted with DEPC water (50 μl) and recovered in an Eppendorf tube which is stored at −80° C.

### RNA Quantification and Quality

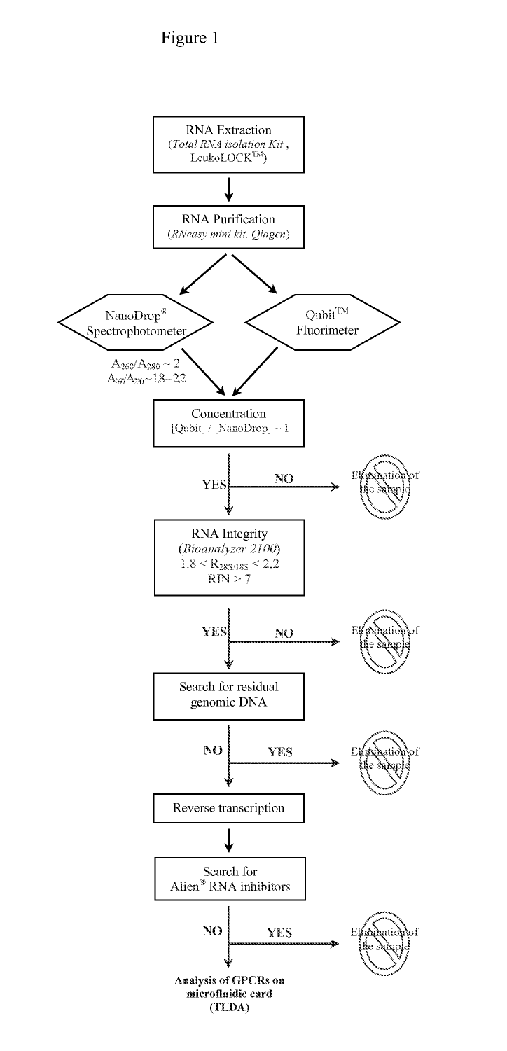

The total RNAs are quantified using two different techniques: 1) highly accurate NanoDrop® spectrophotometry (Thermo Scientific) which enables the analysis of microvolumes (1 μl) resulting in an absorption spectrum from 230 to 350 nm; an RNA of good quality is defined by an A₂₆₀/A₂₈₀ ratio close to 2 and an A₂₃₀/A₂₆₀ ratio of between 1.8 and 2.2; 2) fluorescence measurement (Qubit™ fluorometer, Qiagen) by means of a Quant-it™ RNA assay Kit fluorescent intercalating agent (Qiagen). The result is expressed as RNA concentration proportional to emitted fluorescence. This concentration is compared to that obtained on the NanoDrop®. If the ratio is between 0.9 and 1.1, the RNA is accepted for the rest of the analysis. If this is not the case, the RNA is eliminated (FIG. 1).

### RNA Integrity

The integrity of the RNA is measured by automated capillary electrophoresis (Bioanalyzer 2100, Agilent Technologies) using “RNA 6000 Nano LabChips” (Agilent Technologies). An RNA-binding fluorescent agent is added; the RNA is subjected to an electric field and the fluorescence emitted is measured. The quality of the RNA is evaluated by means of the 28S rRNA/18 S rRNA ratio and by means of the analysis of a complete electrophoregram including the small degraded RNAs, resulting in an RIN (RNA Integrity Number) value. The integrity of the RNA is validated by a 28S/18S ratio>1.8 and an RIN>7 (FIG. 1).

### Contamination By Genomic DNA

Quantitative PCR analysis of the GPCRs requires verification of the absence of residual genomic DNA which could interfere. This validation is carried out by amplification of a highly expressed gene on the RNA samples using a pair of primers designed on a single exon (TagMan® Gene Expression Assay). An absence of amplification during the PCR validates the absence of genomic DNA in the sample. The RNAs which do not give a satisfactory result in this step are eliminated (FIG. 1).

### Reverse Transcription

At the time of the reverse transcription, an experiment for validating the absence of PCR inhibitors in the RNA samples is set up. It involves the introduction of a foreign RNA which does not comprise any homologous eukaryote RNA sequence (Alien®, Stratagene) into the samples. The Alien® RNA (2 μl, 10⁶ copies) is added to the total RNAs (1 μg) having passed the Go-NoGo screen represented on scheme 1 and the latter are subjected to reverse transcription at 37° C. for 2 h in the presence of a reverse transcriptase of viral origin, the Murine Leukaemia Virus reverse transcriptase (MuLV, Life Technologies™). The reaction mixture (sold by Life Technologies™) comprises MuLV, the deoxynucleotide triphosphates, the degenerate primers, and RNase inhibitors in the RT buffer of the kit in a final volume of 100 μl.

### Validation of the Absence of PCR Inhibitors

The real-time PCR amplification is carried out in 96-well plates on an ABI Prism® 7000 machine (Life Technologies). The Alien® cDNA in the presence of cDNA of interest is amplified using Alien® primers (Stratagene). The Alien® amplification threshold cycle (Ct) values are compared in the presence and absence of cDNA samples at various dilutions. The dilution which allows the amplification of the unmodified Alien® cDNA corresponds to a dilution to ⅓ of the sample of cDNA of interest. This dilution corresponds to a sufficient dilution of the sample treatment residues (phenol, ethanol, guanidine, EDTA) acting as PCR inhibitors.

### Quantification of GPCR Gene Expression

The quantification of the GPCR gene expression by real-time quantitative PCR is carried out on an ABI Prism® 7900 machine (Life Technologies™) in a “TagMan® Low Density Array” (TLDA, Life Technologies™) 384-well microfluidic card. Each well of the TLDA card contains a lyophilized TagMan® probe and a pair of specific primers. Among the 384 pairs of primers, 360 are specific for GPCR genes, 20 are specific for housekeeping genes and 4 are specific for a control gene, 18S. The amplification is carried out for 2 h in a final reaction volume of 2 pl. The results of expression of the biomarker gene demonstrated are confirmed by quantitative PCR in a 96-well plate on an ABI Prism® 7000 machine.

### Statistical Analysis and Expression of the Results

The results of the amplifications are provided for each gene as amplification threshold cycle (Ct) value by the RQ Study software integrated into the ABI Prism® 7900 machine.

The statistical analyses are carried out with the Excel spreadsheet and the R software.

### RESULTS AND ANALYSIS

The values of the GPR15 expression in the asthma controls and the COPD controls are not significantly different (p=0.20728197); the two control sets were therefore combined to form the “control” set, thus increasing the analysis power.

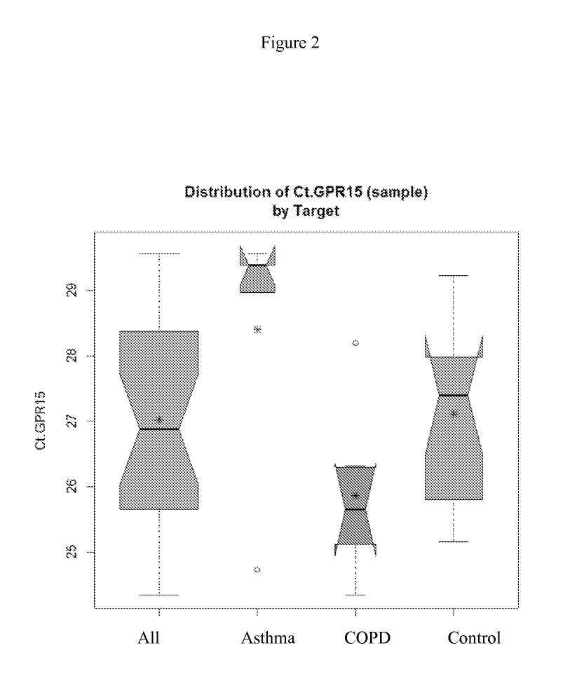

The results of the GPR15 expression are represented graphically: i) as a Boxplot (FIG. 2), ii) as a point-by-point dispersion of the Ct as a function of the age of the patients and a straight line summarizing the variation as a function of age for each group (FIG. 3).

The GPR15 expression values for the COPD patients and the control group are significantly different (p=0.00164019) (FIG. 2).

The GPR15 expression values for the asthmatic patients and the control group are also significantly different (p=0.02535037) (FIG. 2).

The analysis of the GPR15 expression in the control group shows a correlation between the expression and the age of the patient (r²=0.32), as it does in the COPD group (r²=0.51).

FIG. 3 illustrates this dependency of the GPR15 expression value in the various groups of patients with the age variable.

Three atypical values are observed in the COPD group: they involve an asthmatic smoker (A1006), for whom the diagnosis is to be confirmed; a control subject who is a smoker (C2010, 30 packets/year); and a control subject who is a non-smoker (C1001) with a Tiffeneau index (FEV1/FVC %) at 78, and who needs to be reseen.

1-15. (canceled)

16. An in vitro method of diagnosis which makes it possible to determine whether a patient is suffering from asthma or from chronic obstructive pulmonary disease (COPD), in which the G protein-coupled receptor 15 (GPR15) gene expression level is measured in a biological sample from the patient, an underexpression of GPR15 being indicative of asthma and an overexpression of GPR15 being indicative of COPD.

17. The in vitro method according to claim 16, in which the GPR15 gene expression level is measured in a biological sample from the patient, an underexpression of GPR15 being indicative of asthma.

18. The in vitro method according to claim 16, in which the GPR15 gene expression level is compared to a reference expression level.

19. The in vitro method according to claim 18, in which the reference expression level takes into account the age of the patient.

20. The in vitro method according to claim 18, in which the GPR15 gene expression level is measured in a biological sample from the patient and the GPR15 gene expression level is compared to a reference expression level which takes into account the age of the patient, an overexpression of GPR15 being indicative of COPD.

21. The in vitro method according to claim 18, in which the GPR15 gene expression level is determined by measuring the amount of GPR15 protein.

22. The in vitro method according to claim 21, in which the amount of GPR15 protein is measured by immunofluorescence, chemiluminescence, immunohistochemistry, immunocytochemistry, ELISA, the Taqman® protein assay, or a protein or antibody chip.

23. The in vitro method according to claim 21, in which the GPR15 gene expression level is determined by measuring the amount of mRNA encoding the GPR15 protein.

24. The in vitro method according to claim 23, according to which the amount of GPR15 mRNA is measured by means of the real-time quantitative RT-PCR technique, by means of the microfluidic technique, by means of an RNA chip, or by means of LCR, TMA, PCE, bDNA or high-throughput sequencing methods.

25. The in vitro method according to claim 21, in which the biological sample is a blood sample.

26. The in vitro method according to claim 25, in which the GPR15 gene expression product is detected in the leukocytes.
